(19) United States
(12) Patent Application Publication
(10) Pub. No.: US 2016/0045380 A1
(43) Pub. Date: Feb. 18, 2016

(54) WRAPPER FOR AN ABSORBENT ARTICLE

(71) Applicant: First Quality Retail Services, LLC, Great Neck, NY (US)

(72) Inventor: James W. Cree, Loveland, OH (US)

(21) Appl. No.: 14/825,950

(22) Filed: Aug. 13, 2015

Related U.S. Application Data
(60) Provisional application No. 62/036,984, filed on Aug. 13, 2014.

(51) Int. Cl. A61F 13/551; B65D 75/28; B65D 65/38

(52) U.S. Cl. CPC A61F 13/5514; B65D 65/38; B65D 75/28

(57) ABSTRACT

A wrapper for an absorbent article, including a wrapper material including at least one microapertured film layer. The wrapper material has opposite longitudinal sides between first and second ends, and is folded at a first fold axis such that the longitudinal sides are aligned with one another. The wrapper material is bonded along the aligned longitudinal sides to define a pouch having bonded sides and a size for carrying an individual absorbent article therein. The first end of the wrapper material is folded at a second fold axis back over onto a front surface of the pouch to define a flap, the flap having opposite sides bonded with the pouch sides.

### RELATED APPLICATION

This application is a non-provisional based on U.S. Provisional Application No. 62/036,984, filed Aug. 13, 2014, the contents of which are incorporated herein by reference in their entirety.

### FIELD

The present invention relates to wrappers for absorbent articles, and more particularly, to wrappers made from a film layer.

### BACKGROUND

For convenience, discreetness and protection, some disposable absorbent articles such as bladder control pads, pantiliners and sanitary napkins are packaged individually within a larger sized bag or box. The individual packages or wrappers for these products provide a convenient means for a user to carry an individual product or two in her pocketbook or purse. The wrapper provides protection for the hygienic pad from contaminants that could otherwise contact the product. Typically these hygienic pads have an adhesive on one side so that the pad may be secured to the user's underwear.

Most of the wrappers seen on the market today consist of a polyethylene film wrapped around an absorbent pad that is bi-folded or tri-folded. The film is sealed along two of its edges and generally is sealed by adhesive in one spot that occurs centrally across the open seam between the sealed edges. Upon opening the seal along the seam, the pad can be removed from the wrapper. Then, a siliconized release paper is removed from the adhesive strip on the pad, exposing the adhesive which enables the user to secure the absorbent pad to her underwear to keep the pad in place.

Although such film wrappers do provide protection for the absorbent pad, the film wrapper will crinkle when handled, and this noise diminishes the discreetness of the product.

### SUMMARY OF THE INVENTION

An object of the present invention is to provide a discreet, quiet, soft and convenient wrapper material for individual wrapping of absorbent articles.

According to an exemplary embodiment of the present invention, a wrapper for an absorbent article comprises: a wrapper material comprising at least one microapertured film layer; the wrapper material having opposite longitudinal sides between first and second ends; the wrapper material folded at a first fold axis such that longitudinal sides are aligned with one another, and the wrapper material bonded along the aligned longitudinal sides to define a pouch having bonded sides and a size for carrying an individual absorbent article therein, the first end of the wrapper material folded at a second fold axis back over onto a front surface of the pouch to define a flap, the flap having opposite sides bonded with the pouch sides.

In at least one embodiment, upon the wrapper material being folded at a first fold axis, the at least one microapertured film layer is outwardly disposed.

In at least one embodiment, upon the wrapper material being folded at a first fold axis, the at least one microapertured film layer is inwardly disposed.

In at least one embodiment, the wrapper material is a two-layer microapertured film.

In at least one embodiment, the wrapper material is a three-layer microapertured film.

In at least one embodiment, the wrapper material comprises at least two microapertured film layers, and one of the microapertured film layers is colored differently from the other ones of the microapertured film layers.

In at least one embodiment, microapertures within the at least one microapertured film layer forms a pattern.

In at least one embodiment, the wrapper material further comprises a nonwoven layer.

Other features and advantages of embodiments of the invention will become readily apparent from the following detailed description, the accompanying drawings and the appended claims.

### BRIEF DESCRIPTION OF THE DRAWINGS

The features and advantages of exemplary embodiments of the present invention will be more fully understood with reference to the following, detailed description when taken in conjunction with the accompanying figures, wherein:

FIG. 1 is a perspective view showing a wrapper according to an exemplary embodiment of the present invention as used with an absorbent article;

FIG. 2 is a perspective view of the wrapper of FIG. 1 as shown in its opened state; and

FIG. 3 is a cross-sectional view taken along the line 3-3 indicated in FIG. 1.

### DETAILED DESCRIPTION

The headings used herein are for organizational purposes only and are not meant to be used to limit the scope of the description or the claims. As used throughout this application, the words “may” and “can” are used in a permissive sense (i.e., meaning having the potential to), rather than the mandatory sense (i.e., meaning must). Similarly, the words “include,” “including,” and “includes” mean including but not limited to. To facilitate understanding, like reference numerals have been used, where possible, to designate like elements common to the figures.

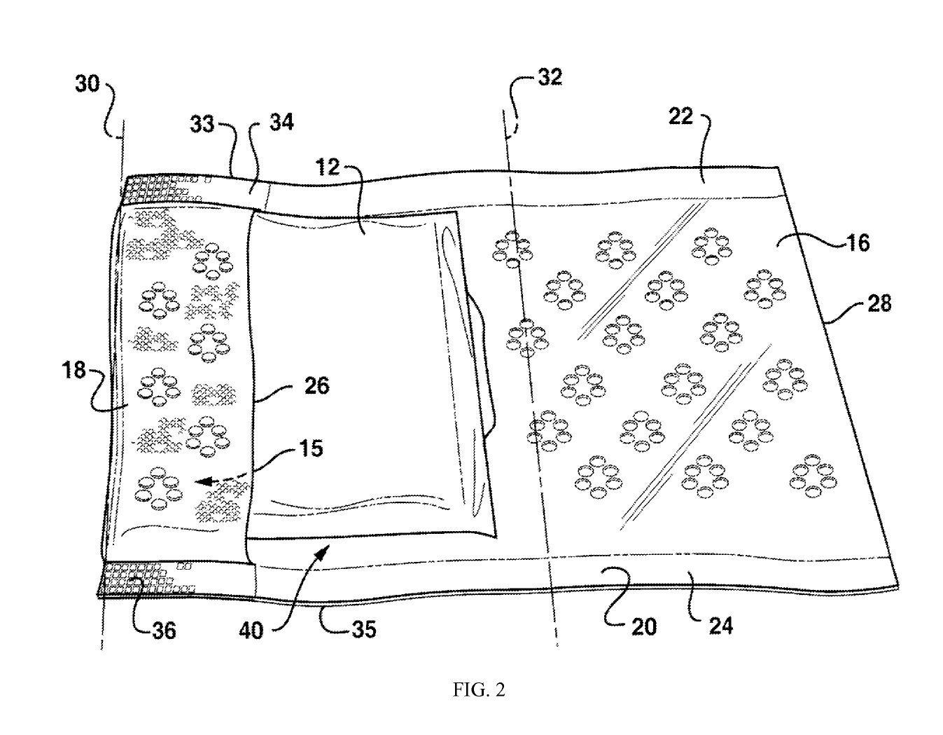

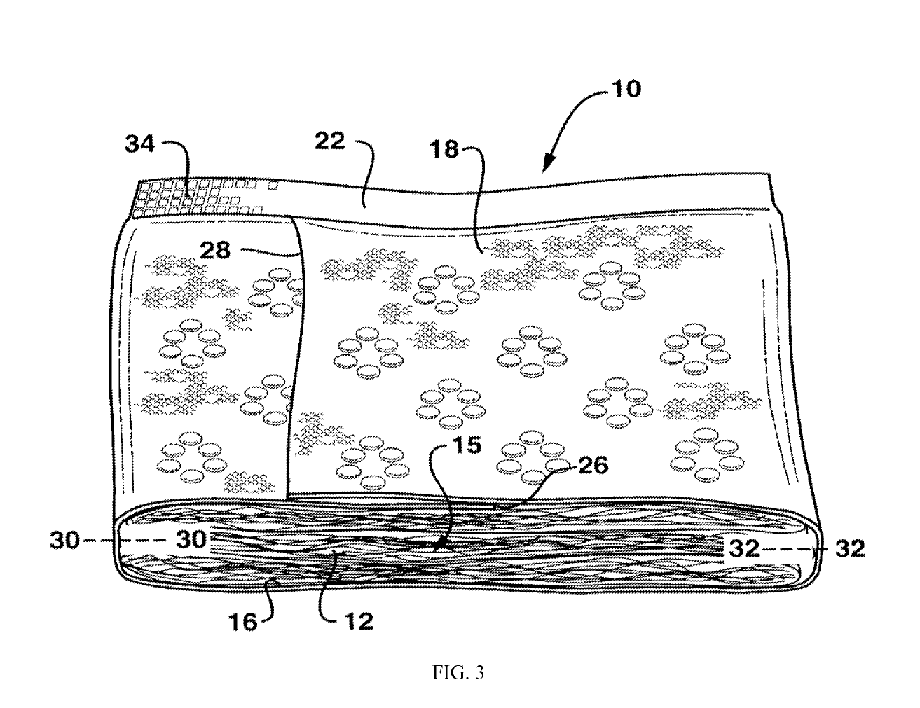

Referring to the figures, an embodiment of an individually wrapped absorbent article package 10 is generally illustrated. An absorbent article 12 is carried in the package 10, as shown in FIG. 2. The invention is not limited to any particular type of absorbent article. For example, the absorbent article 12 may be a catamenial device such as a sanitary napkin, a panty liner, a labial pad, an incontinence pad, or any other type of absorbent article which can be used to absorb menstrual fluid, urine, body fluid, body exudate, etc. A detailed description of such absorbent articles is not necessary for purposes of the present invention. For purposes of describing the invention only, the absorbent article 12 is shown and referred to herein as a sanitary pad or napkin. The absorbent article 12 may be folded in any desired pattern to fit in the package 10. In the illustrated embodiment, the absorbent article 12 is in a tri-fold pattern, as seen in FIG. 3.

The package 10 includes an elongate piece of wrapper material, generally 14, folded and bonded into a pouch configuration. For example, the wrapper material 14 may be an elongated rectangular piece having a first end 26, an opposite second end 28, and generally parallel longitudinal sides 33 and 35 extending between the ends 26 and 28.

The invention is not limited to any particular type of pouch configuration. Various pouch configurations are known and used in the art for individually packaging feminine care absorbent articles and any such configuration may be used in a package according to the invention. The unique features of the present wrapper material 14 will provide a benefit to any pouch configuration.

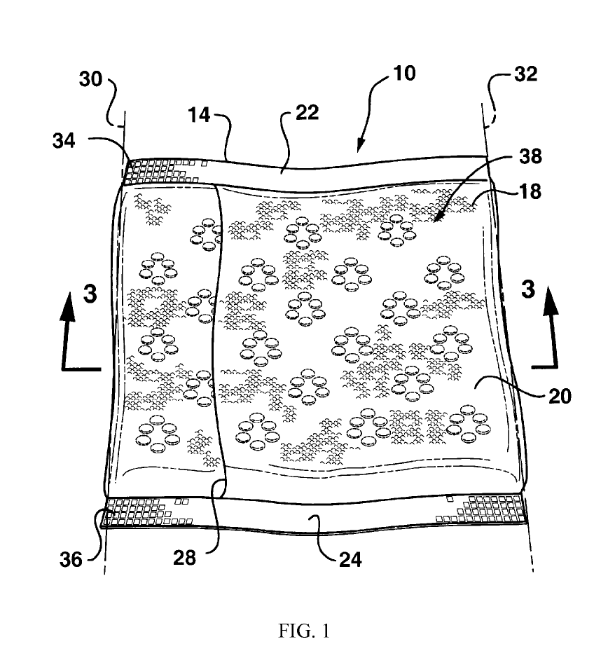

Referring to FIG. 2, it can be seen that the wrapper material is essentially folded around the absorbent article 12 such that the pouch 15 is formed around the article. The wrapper material 14 is first folded at a first fold axis 30 such that the first end 26 is folded towards but spaced from the second end 28, as can be seen particularly in FIGS. 2 and 3. The distance between the first end 26 and second end 28 may vary depending on the desired length of a resulting flap 20, as described below. The aligned longitudinal sides of the wrapper material 14 define sides 34 and 36 of the pouch 15. The second end 28 of the wrapper material is then folded at a second fold axis 32 so as to extend back over the first end 26 and thus defines the flap 20 that closes off the pouch 15, as particularly seen in FIG. 1. The flap 20 has longitudinal sides 24 and 22 that align with the material sides 33 and 35 and pouch sides 34 and 36. The sides of the package 10 are then bonded in a conventional manner, for example with a heat/pressure embossing roll. The flap sides 22 and 24 are bonded to the material sides 33 and 35 and pouch sides 34 and 36 in a single pass operation. It may be the case that the first end 26 of the wrapper material 14 extends essentially to the second fold axis 32 and, thus, the flap sides 22 and 24 would be bonded along their entire length to pouch sides 34 and 36.

Referring to FIGS. 1 and 3, it can be seen that the edge of the second end 28 extends across the front surface of the pouch 15. It may be desired to adhere all or a portion of this edge to the pouch surface. However, in a desirable embodiment of the package according to the invention, this edge is left un-adhered to the pouch between its bonded sides 22 and 24.

The wrapper material 14 according to an exemplary embodiment of the present invention is a microapertured polymeric film, such as the film described in U.S. Pat. No. 5,158,819, the contents of which are incorporated herein by reference in their entirety. When used as the outer exposed surface of the wrapper 10, such films provide the wrapper 10 with a soft and silky tactile impression and also reduce or eliminate the noise that is otherwise present during use of conventional wrappers. Further, the microapertured structure of the film allows for the formation of a contrasting visually discernable three-dimensional pattern that imparts an embossed appearance. Such three-dimensional patterns formed on the wrapper material 14 may be, for example, a logo, brand name or other type of design. This eliminates the need to form such patterns by printing during the wrapper manufacturing process.

The wrapper material 14 may be a multi-layer polymeric film made by a coextrusion process. The film may be a two-layer or three-layer film that is microapertured after the coextrusion process to form the wrapper material 14. The individual layers of the multi-layer film may differ in color to provide a strong visual signal. For example, in the case of a three-layer polymeric film, the middle layer or one of the outer layers may have a different color than the other layers.

The polymeric film used to form the wrapper material 14 include films made from polyolefin resins such as polythelene, polyproplene and metalocene variant of PE and PP. In addition the film can be made with resin blends containing poly(vinyl alcohol), polyvinyl acetate, ethylene vinyl alcohol, polyurethane, ethylene methyl acrylate, and ethylene methyl acrylic acid. If desired, it is also possible to add fillers to the film such as, for example, calcium carbonate and titanium dioxide.

In an exemplary embodiment, the wrapper material 14 may include a nonwoven layer that is laminated or otherwise attached to the microapertured polymeric film layers. The nonwoven layer may form the base layer of the wrapper material 14. The film layers and nonwoven layer may be laminated together by any lamination technique known to those skilled in the art. Suitable lamination means include, but are not limited to, adhesives, ultrasonic bonding and thermomechanical bonding as through the use of heated calendering rolls. Such calendering rolls will often include a patterned roll and a smooth anvil roll, though both rolls may be patterned or smooth and one, both or none of the rolls may be heated. The nonwoven layer may be made of, for example, a meltblown web, a spunbond web, an SMS web, a thermobonded carded web, an airlaid web or any other type of nonwoven web material. The fibers may be made of polytheylene, polypropylene and/or bicomponents thereof.

While particular embodiments of the invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A wrapper for an absorbent article, comprising:
a wrapper material comprising at least one microapertured film layer;
the wrapper material having opposite longitudinal sides between first and second ends;
the wrapper material folded at a first fold axis such that longitudinal sides are aligned with one another, and the wrapper material bonded along the aligned longitudinal sides to define a pouch having bonded sides and a size for carrying an individual absorbent article therein, the first end of the wrapper material folded at a second fold axis back over onto a front surface of the pouch to define a flap, the flap having opposite sides bonded with the pouch sides.

2. The wrapper of claim 1, wherein, upon the wrapper material being folded at a first fold axis, the at least one microapertured film layer is outwardly disposed.

3. The wrapper of claim 1, wherein, upon the wrapper material being folded at a first fold axis, the at least one microapertured film layer is inwardly disposed.

4. The wrapper of claim 1, wherein the wrapper material is a two-layer microapertured film.

5. The wrapper of claim 1, wherein the wrapper material is a three-layer microapertured film.

6. The wrapper of claim 1, wherein the wrapper material comprises at least two microapertured film layers, and one of the microapertured film layers is colored differently from the other ones of the microapertured film layers.

7. The wrapper of claim 1, wherein microapertures within the at least one microapertured film layer forms a pattern.

8. The wrapper of claim 1, wherein the wrapper material further comprises a nonwoven layer.
